(19) United States
(12) Patent Application Publication
(10) Pub. No.: US 2022/0211309 A1
(43) Pub. Date: Jul. 7, 2022

(54) Disposable Lancing Device and Method of Operating the Same

(71) Applicant: Medifun Corporation, Taichung (TW)

(72) Inventor: William Chen, Taichung (TW)

(21) Appl. No.: 17/542,737

(22) Filed: Dec. 6, 2021

(30) Foreign Application Priority Data
Jan. 4, 2021 (TW) 110100151

(51) Int. Cl. A61B 5/151; A61B 5/15

(52) U.S. Cl. CPC A61B 5/15176; A61B 5/150022; A61B 5/15117; A61B 5/150725; A61B 5/150885; A61B 5/150282

(57) ABSTRACT

A disposable lancing device contains: a first casing, a second casing, and a needle holder. The first casing includes a close segment, an open segment, a slideway, two tilted contact faces, a fixing portion, two recesses, and two push fringes. The second casing includes an accommodation chamber, two limitation extensions, and multiple drive protrusions. A forcing face is defined on the second casing opposite to the two limitation extensions, and a through hole is defined on a center of the forcing face. The through hole has a protruded rib. The needle holder includes a collection needle having a syringe needle, and the needle holder further includes a trigger spring, a return spring, and two symmetrical flexible wings. The two symmetrical flexible wings include two driving segments configured to abut against the two symmetrical flexible wings to flexibly retract inward by using the two tilted contact faces.

### FIELD OF THE INVENTION

The present invention relates to a disposable lancing device and a method of operating the same.

### BACKGROUND OF THE INVENTION

In current general medical examination process, injection or invasive blood sampling devices are often used. For example, in order to effectively control the blood glucose concentration of diabetic patients, medical staff must use the blood sampling device to prick the patient's skin to collect blood samples every day. Drop the collected blood sample on the test piece, insert the test piece into the blood glucose meter, and read the blood glucose concentration of the blood sample.

At present, there are quick and easy-to-operate testing instruments on the market. Only a small amount of blood from the subject can be detected. Therefore, the subject only needs to puncture the skin and squeeze out a small amount of blood, which will be sufficient for the detection of the testing instrument. Therefore, there are also blood collection devices on the market designed to meet this demand. One type of blood collection device mainly has a pen-shaped grip and uses a pressing method to make the blood sampling needle puncture the skin of the person being collected. After the needle is used, replace the needle. However, this method may be caused by the negligence of the blood sampling staff forgetting to replace the blood sampling needle. The blood sampler suffers from infection, and the risk of the blood sampler being punctured by the lancet when replacing the lancet is also increased.

With reference to FIGS. 14-17, a disposable lancing device is disclosed in TW Patent No. 094141724 and contains: a casing A10 in which two abutting portions A11 are formed, an opening A12 defined on a front end of the casing A10, multiple locking orifices A13 defined a rear end of the casing A10, such that a needle holder A20 is received in the casing A10. The needle holder A20 includes a collection needle A22 formed on an end thereof corresponding to the opening A12, and the collection needle A22 is fitted with a protective tube A40. The needle holder A20 includes two flexible wings A21 extending from two sides thereof and two free segments of the two flexible wings A21 abut against the two abutting portions A11. The needle holder A20 further includes a column A23 fitted with a spring A30, and a distal end of the spring A30 engages with a press member A14 which is movably mounted on the distal end of the casing A10. The press member A14 has two protrusions A15 extending from an outer wall thereof and corresponding to the multiple locking orifices A13 of the casing A10. The two protrusions A15 are engaged in the multiple locking orifices A13. When desiring to collect blood, the protective tube A40 is removed, as illustrated in FIG. 14, and the press member A14 is pressed so that the two protrusions A15 are removed from the multiple locking orifices A13 to push the two flexible wings A12 of the needle holder A20 to retract inward, and the two free segments of the two flexible wings A21 detach from the two abutting portions A11, as shown in FIG. 15, wherein the spring A30 is released to urge the needle holder A20 to trigger the collection needle A22 into a skin of a user, as shown in FIG. 16, and the needle holder A20 is pushed by the spring A30 to retract into the casing A10.

However, the spring A30 forces the needle holder A20 to trigger the collection needle A22 into a skin of a user suddenly, thus causing stabbing pain. Furthermore, the two free segments of the two flexible wings A21 of the needle holder A20 friction the casing A10 to cause inaccurate trigger of the collection needle A22 and damage to the casing A10.

The present invention has arisen to mitigate and/or obviate the afore-described disadvantages.

### SUMMARY OF THE INVENTION

The primary objective of the present invention is to provide a disposable lancing device that is capable of overcoming the shortcomings of the conventional disposable lancing device.

To obtain above-mentioned aspect, a disposable lancing device provided by the present invention contains: a first casing, a second casing, and a needle holder.

The first casing includes a close segment formed on a first end of the first casing, an open segment formed on a second end of the first casing, a slideway formed in the first casing, two tilted contact faces formed on two side wall of the slideway, two first shoulders formed on the slideway, a fixing portion formed on the slideway and corresponding to the close segment, two recesses defined on two side walls of the first casing, and two push fringes formed around the two recesses.

The second casing includes an accommodation chamber defined in the second casing, and the second casing is slidably received on the slideway of the first casing, the second casing includes two limitation extensions corresponding to the first casing and engaged with the two first shoulders of the casing, multiple drive protrusions proximate to the two limitation extensions and having multiple urge segments formed on multiple predetermined positions of the multiple drive protrusions. A forcing face is defined on an end of the second casing opposite to the two limitation extensions, and a through hole is defined on a center of the forcing face. The through hole has a protruded rib extending outward therearound opposite to the forcing face.

The needle holder is defined between the first casing and the second casing, and the needle holder includes a collection needle accommodated in the needle holder. The collection needle includes a syringe needle fixed on an end thereof and corresponding to the through hole of the second casing, the needle holder further includes a trigger spring corresponding to the fixing portion of the first casing, and the needle holder further includes a return spring corresponding to the through hole of the second casing. The needle holder includes two symmetrical flexible wings corresponding to the two tilted contact faces of the first casing, and the two symmetrical flexible wings include two driving segments formed on two distal ends thereof. The two driving segments abut against the two symmetrical flexible wings of the needle holder to flexibly retract inward by using the two tilted contact faces of the first casing.

Thereby, the needle holder includes the trigger spring and the return spring so that when the trigger spring pushes the needle holder toward the through hole of the second casing, the return spring buffers the pushing force of the trigger spring to the syringe needle so as to reduce the stabbing pain. Preferably, when the forcing face presses the collection portion by mating with the protruded rib, the subcutaneous fat of the collection portion is pressed in the thickness so as to reduce the stabbing pain when piercing the collection needle into the skin of the user. In addition, the trigger spring and the return spring pull the needle holder and the syringe needle back quickly to finish the blood collection.

### BRIEF DESCRIPTION OF THE DRAWINGS

FIG. 1 is a perspective view showing the assembly of a disposable lancing device according to a preferred embodiment of the present invention.

FIG. 2 is a perspective view showing the operation of the disposable lancing device according to the preferred embodiment of the present invention.

FIG. 3 is a side plane view showing the operation of the disposable lancing device according to the preferred embodiment of the present invention.

FIG. 4 is a cross-sectional perspective view showing the assembly of the disposable lancing device according to the preferred embodiment of the present invention.

FIG. 5 is another cross-sectional perspective view showing the assembly of the disposable lancing device according to the preferred embodiment of the present invention.

FIG. 6 is a perspective view showing the exploded components of the disposable lancing device according to the preferred embodiment of the present invention.

FIG. 7 is a cross-sectional perspective view showing the exploded components a part of the disposable lancing device according to the preferred embodiment of the present invention.

FIG. 8 is a cross sectional view showing the assembly of the disposable lancing device according to the preferred embodiment of the present invention.

FIG. 9 is a cross sectional view showing the operation of the disposable lancing device according to the preferred embodiment of the present invention.

FIG. 10 is another cross sectional view showing the operation of the disposable lancing device according to the preferred embodiment of the present invention.

FIG. 11 is also another cross sectional view showing the operation of the disposable lancing device according to the preferred embodiment of the present invention.

FIG. 12 is still another cross sectional view showing the operation of the disposable lancing device according to the preferred embodiment of the present invention.

FIG. 13 is a flow chart of a method of operating the disposable lancing device according to the preferred embodiment of the present invention.

FIG. 14 is a cross sectional view showing the assembly of a conventional disposable lancing device.

FIG. 15 is a cross sectional view showing the operation of the conventional disposable lancing device.

FIG. 16 is another cross sectional view showing the operation of the conventional disposable lancing device.

FIG. 17 is also another cross sectional view showing the operation of the conventional disposable lancing device.

### DETAILED DESCRIPTION OF THE PREFERRED EMBODIMENTS

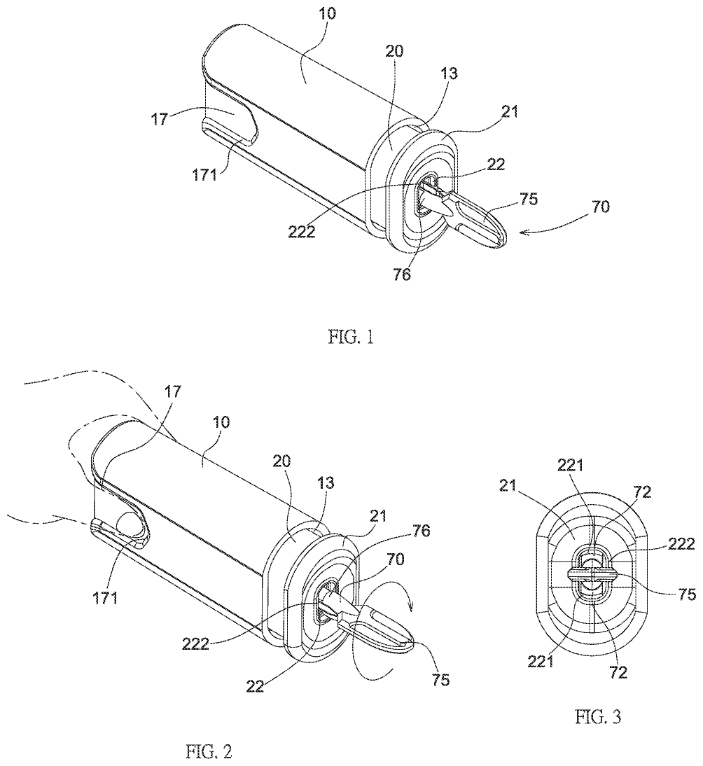

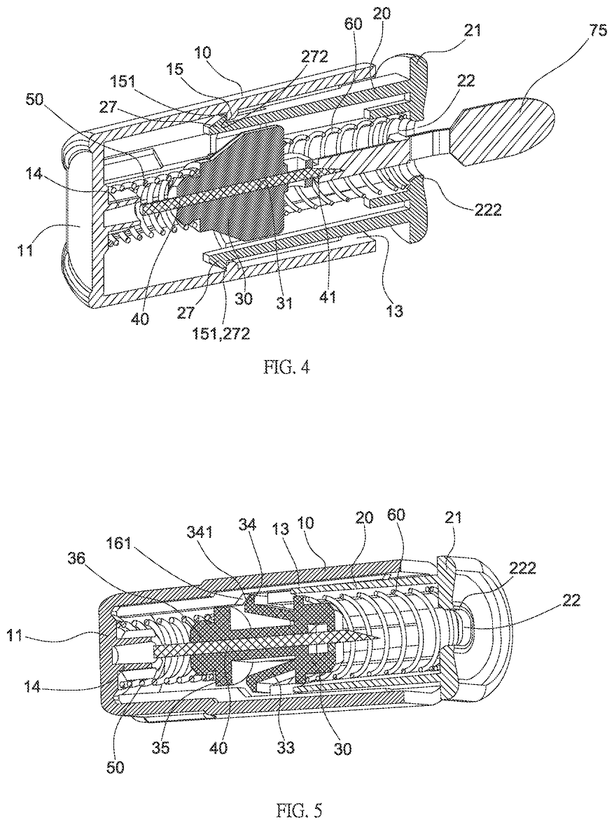

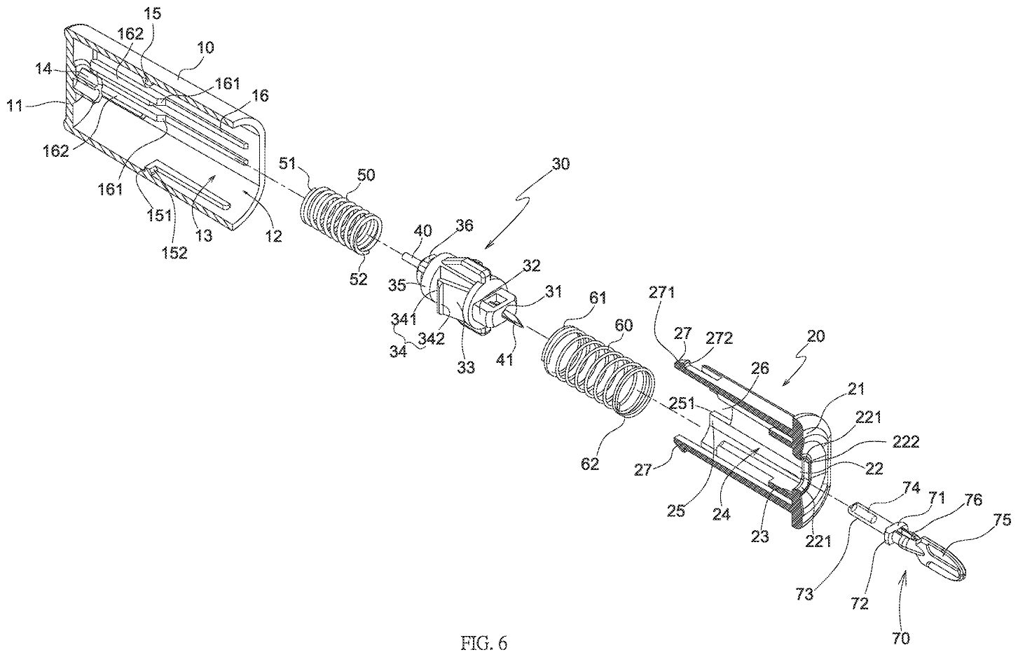

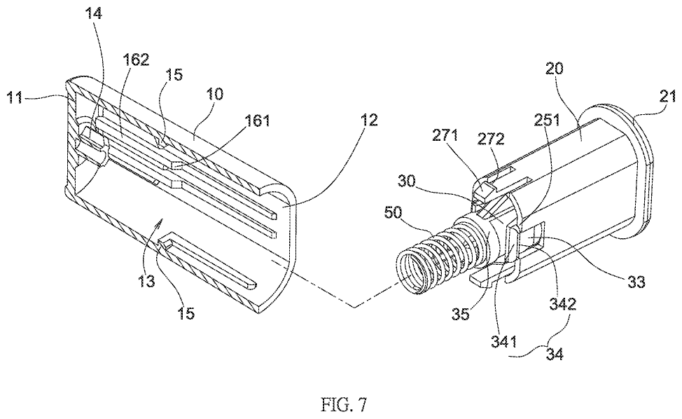

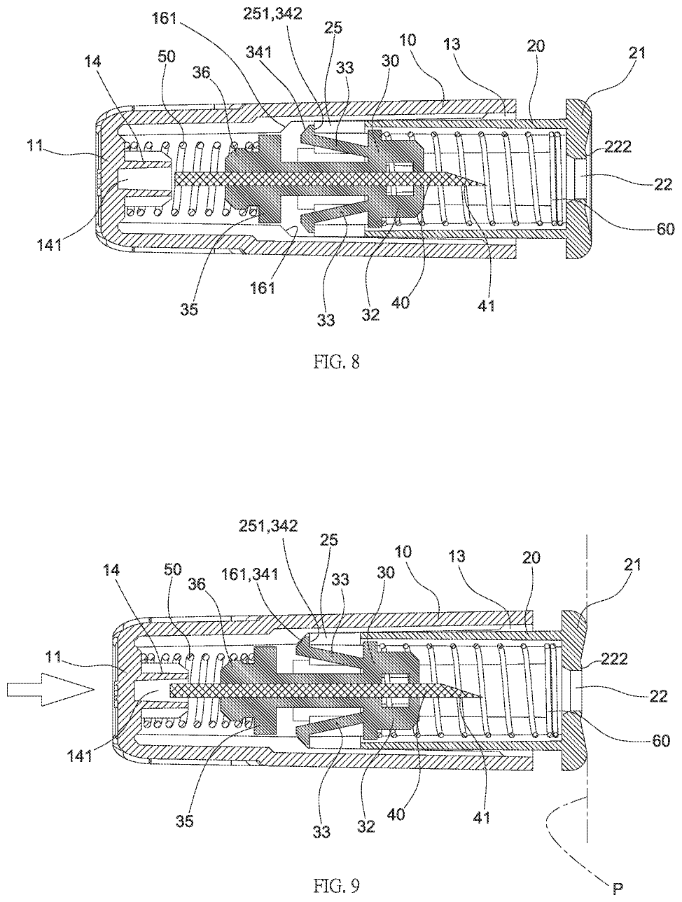

With reference to FIGS. 1-12, a disposable lancing device according to a preferred embodiment of the present invention comprises:

a first casing 10 including a close segment 11 formed on a first end of the first casing 10, an open segment 12 formed on a second end of the first casing 10, a slideway 13 formed in the first casing 10, two tilted contact faces 161 formed on two side wall of the slideway 13, two abutting ribs 16 extending from two low ends of the two tilted contact faces 161, and two slide tracks 162 formed on two high ends of the two tilted contact faces 161; two first shoulders 15 formed on the slideway 13 between the two tilted contact faces 161 and the two slide tracks 162, two guide faces 152 formed on the two first shoulders 15, and two first stop faces 151 formed on the two first shoulders 15 opposite to the two guide faces 152, a fixing portion 14 formed on the slideway 13 and corresponding to the close segment 11, two recesses 17 defined on two side walls of the first casing 10, and two push fringes 171 formed around the two recesses 17, as shown in FIG. 2, such that when a user presses the two recesses 17, the first casing 10 does not move by contacting fingers with the two push fringes 171;

a second casing 20 including an accommodation chamber 24 defined in the second casing 20, and the second casing 20 being slidably received on the slideway 13 of the first casing 10, the second casing 20 further including two limitation extensions 27 corresponding to the first casing 10 and engaged with the two first shoulders 15 of the casing 10, wherein the two limitation extensions 27 have two first inclined guide faces 271 formed thereon and corresponding to the two guide faces 152 of the two first shoulders 15, and the two limitation extensions 27 have two second stop faces 272 formed thereon opposite to the two first inclined guide faces 271, such that the two first inclined guide faces 271 of the two limitation extensions 27 abut against the two guide faces 152 of the two first shoulders 15, and the two second stop faces 272 of the two limitation extensions 27 move to the two first stop faces 151 of the two first shoulders 15 so that the first casing 10 limits with and slides relative to the second casing 20; the second casing 20 further including multiple drive protrusions 25 proximate to the two limitation extensions 27 and having multiple urge segments 251 formed on multiple predetermined positions of the multiple drive protrusions 25, and the second casing 20 including multiple shrinkage trenches 26, wherein a respective one shrinkage trench 26 is defined between any two adjacent drive protrusions 25, a forcing face 21 is defined on an end of the second casing 20 opposite to the two limitation extensions 27, and a through hole 22 is defined on a center of the forcing face 21, wherein the through hole 22 has a protruded rib 222 extending outward therearound opposite to the forcing face 21, such that when the forcing face 21 presses a collection portion P by mating with the protruded rib 222, a subcutaneous fat of the collection portion P is pressed in a thickness so as to reduce a stabbing pain when piercing the disposable lancing device into a skin of the user;

a needle holder 30 defined between the first casing 10 and the second casing 20, and the needle holder 30 including a receiving orifice 31 and configured to accommodate a collection needle 40, wherein the collection needle 40 includes a syringe needle 41 fixed on an end thereof and corresponding to the through hole 22 of the second casing 20, the needle holder 30 further includes a trigger spring 50 corresponding to the fixing portion 14 of the first casing 10, and the fixing portion 14 has a connecting orifice 141 defined therein, the needle holder 30 further includes a return spring 60 corresponding to the through hole 22 of the second casing 20; wherein the needle holder 30 further includes a connection projection 35 formed on an end thereof and corresponding to the trigger spring 50, a first fitting portion 36 extending from an end of the connection projection 35, a triggering segment 52 formed on an end of the trigger spring 50 and corresponding to the first fitting portion 36, such that the triggering segment 52 is fixed on the first fitting segment 36, and the trigger spring 50 includes a positioning segment 51 formed on an end thereof and corresponding to the fixing portion 14 of the first casing 10, such that the positioning segment 51 is mounted on the fixing portion 14 of the first casing 10; the needle holder 30 further includes a second fitting portion 32 formed on an end thereof corresponding to the return spring 60, and the return spring 60 includes a returning segment 61 extending from a first end thereof and corresponding to the second fitting portion 32, such that the returning segment 61 is fixed on the second fitting portion 32, and the return spring 60 includes a locating segment 62 extending from a second end thereof and corresponding to the accommodation chamber 24 of the second casing 20, the accommodation chamber 24 of the second casing 20 has a disposing face 23 corresponding to the locating segment 62 so that the locating segment 62 is fixed on the disposing 23 of the second casing 20; and the needle holder 30 further includes two symmetrical flexible wings 33 corresponding to the two tilted contact faces 161 of the first casing 10, wherein the two symmetrical flexible wings 33 include two driving segments 34 formed on two distal ends thereof, the two driving segments 34 have two second inclined guide faces 341 formed on two first ends thereof, and the two driving segments 34 have two pushed faces 342 formed on two second ends thereof opposite to the two second inclined guide faces 341, wherein the second casing 20 includes the multiple shrinkage trenches 26, and the respective one second shrinkage trench 26 is defined between any two adjacent drive protrusions 25, such that when the second casing 20 is moved, the multiple second shrinkage trenches 26 shrink the two tilted contact faces 161 and the two slide tracks 162 of the first casing 10 so that when the multiple urge segments 251 of the second casing 20 push the two pushed faces 342 of the needle holder 30, the two second inclined guide faces 341 are pushed horizontally by the two tilted contact faces 161 of the first casing 10, the two symmetrical flexible wings 33 of the needle holder 30 are pushed to flexibly retract inward, and the two tilted contact faces 161 and the two slide tracks 162 of the first casing 10 move into the multiple shrinkage trenches 26 of the second casing 20, wherein the two driving segments 34 abut against the two symmetrical flexible wings 33 of the needle holder 30 to flexibly retract inward by using the two tilted contact faces 161 of the first casing 10.

The disposable lancing device further comprises a safety sleeve 70 configured to receive the syringe needle 41 of the collecting needle 40 of the needle holder 30, wherein the safety sleeve 70 includes a connecting segment 73 formed on a first end thereof, a slot 74 defined in the connecting segment 73 and configured to receive the syringe needle 41, and a grip portion 75 formed on a second end of the safety sleeve 70 and extending out of the through hole 22; wherein a stopping portion 71 is defined between the connecting segment 73 and the grip portion 75, a second shoulder 72 is defined between the connecting segment 73 and the grip portion 75 of the safety sleeve 70, and the second shoulder 71 has two extending tabs 72 extending from two sides of the second shoulder 71, wherein the two extending tabs 72 have a limiting rib 76 facing the grip portion 75, and the second casing 20 further includes an elongated groove 221 defined in the through hole 22 of the second casing 20 and corresponding to the limiting rib 76 and the two extending tabs 72 so that the limiting rib 76 and the two extending tabs 72 rotate an angle (such as 90 degrees) by using the grip portion 75, and the safety sleeve 70 is engaged in the through hole 22 of the second casing 20. Alternatively, the grip portion 75 rotates an angle so that the limiting rib 76 and the two extending tabs 72 are aligned with the elongated groove 221 of the through hole 22, and the safety sleeve 70 moves into and out of the through hole 22, wherein the two extending tabs 72 are configured to avoid a removal of the safety sleeve 70 from the collecting needle 40 and extending the syringe needle 41. Furthermore, the limiting rib 76 is configured to avoid a careless trigger of the safety sleeve 70 toward the collecting needle 40, thus obtaining using safety by using the limiting rib 76 and the two extending tabs 72 relative to the through hole 22 of the second casing 20.

Referring to FIGS. 1-3, when collecting blood, the grip portion 75 of the safety sleeve 70 is rotated to an angle so that the two extending tabs 72 are aligned with the elongated groove 221 of the through hole 22, and the safety sleeve 70 is removed from the syringe needle 41.

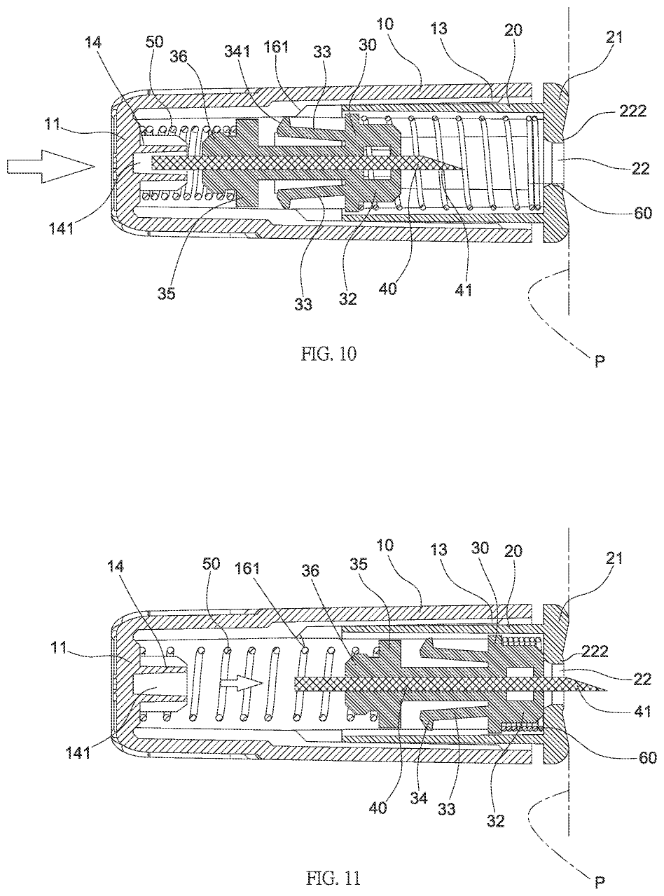

As illustrated in FIG. 9, when the user is desired to collect the blood on the collection portion P, the forcing face 21 of the second casing 20 abuts against the collection portion P, and the first casing 10 is pushed toward the collection portion P, wherein the multiple urge segments 251 of the second casing 20 push the two pushed faces 342 of the needle holder 30, the two second inclined guide faces 341 of the needle holder 30 contact with the two tilted contact faces 161 of the first casing 10, the connection projection 35 presses the trigger spring 50, and the collecting needle 40 is inserted into the connecting orifice 141.

Referring further to FIGS. 10 and 11, when the two second inclined guide faces 341 are pushed horizontally by the two tilted contact faces 161 of the first casing 10, the two symmetrical flexible wings 33 of the needle holder 30 are pushed to flexibly retract inward, and the two tilted contact faces 161 and the two slide tracks 162 of the first casing 10 move into the multiple shrinkage trenches 26 of the second casing 20, wherein the two driving segments 34 abut against the two symmetrical flexible wings 33 of the needle holder 30 so that the two symmetrical flexible wings 33 flexibly retract inward by using the two tilted contact faces 161 of the first casing 10. In the meantime, the trigger spring 50 is released to push the needle holder 30 toward the through hole 22 of the second casing 20, and the syringe needle 41 extends out of the through hole 22 of the second casing 20 and pierces into the collection portion P, wherein the return spring 60 buffers a pushing force of the trigger spring 50 to the syringe needle 41 so as to reduce the stabbing pain.

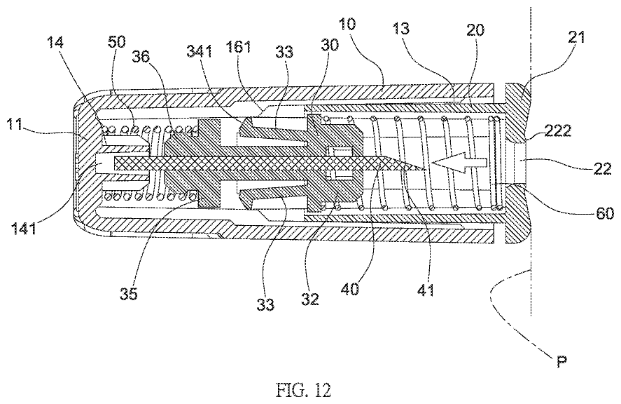

With reference to FIG. 12, when the trigger spring 50 recovers to a unforced state, the trigger spring 50 and the return spring 60 pull the needle holder 30 and the syringe needle 41 back quickly to finish the blood collection, and the syringe needle 41 is hidden in the first casing 10 and the second casing 20 to avoid contacting with the user.

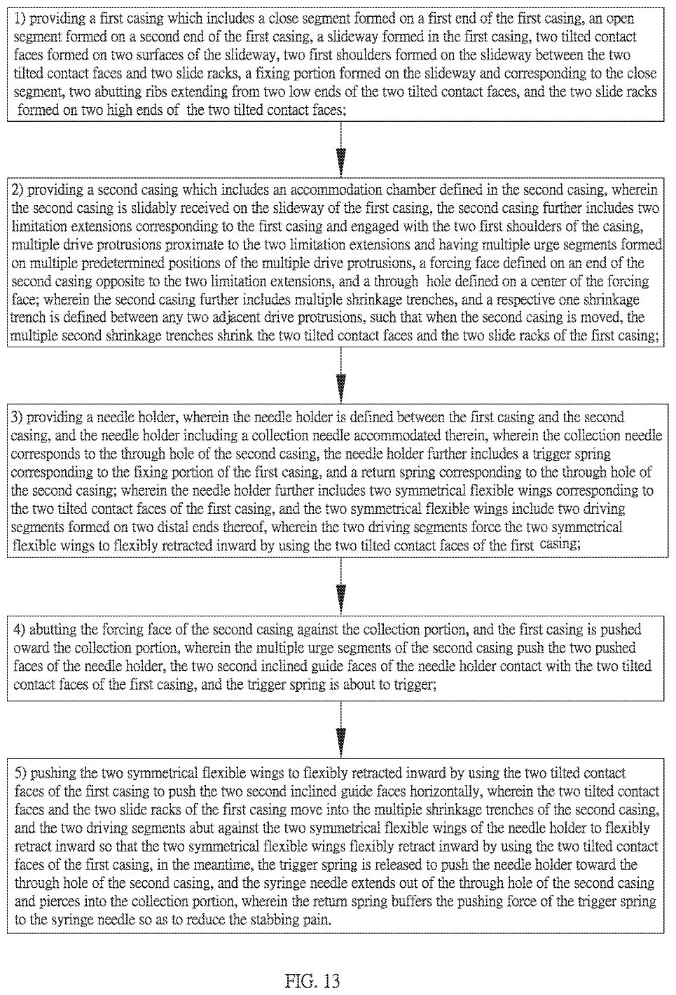

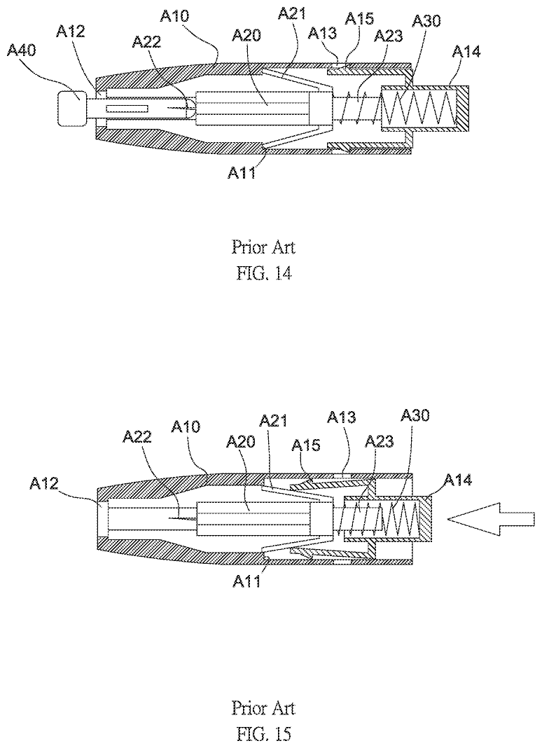

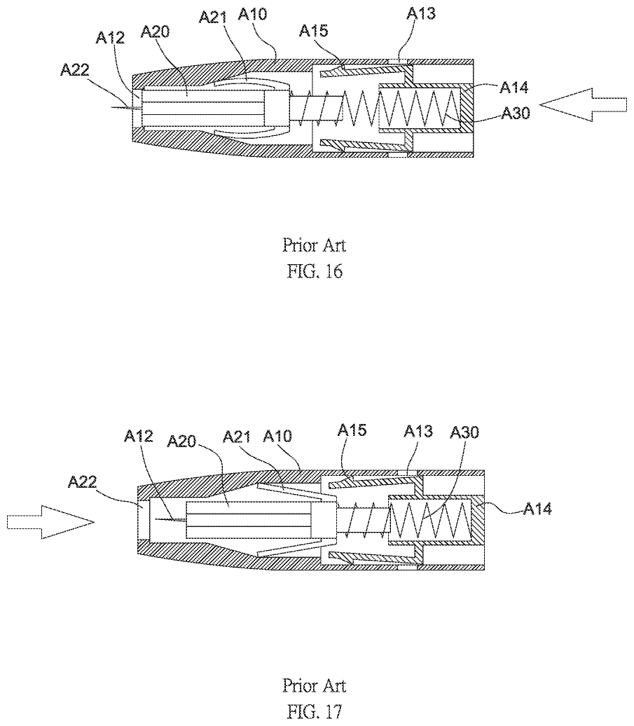

With reference to FIG. 13, a method of operating the disposable lancing device comprises steps of:

1) providing a first casing 10 which includes a close segment 11 formed on a first end of the first casing 10, an open segment 12 formed on a second end of the first casing 10, a slideway 13 formed in the first casing 10, two tilted contact faces 161 formed on two surfaces of the slideway 13, two first shoulders 15 formed on the slideway 13 between the two tilted contact faces 161 and two slide tracks 162, a fixing portion 14 formed on the slideway 13 and corresponding to the close segment 11, two abutting ribs 16 extending from two low ends of the two tilted contact faces 161, and the two slide tracks 162 formed on two high ends of the two tilted contact faces 161;

2) providing a second casing 20 which includes an accommodation chamber 24 defined in the second casing 20, wherein the second casing 20 is slidably received on the slideway 13 of the first casing 10, the second casing 20 further includes two limitation extensions 27 corresponding to the first casing 10 and engaged with the two first shoulders 15 of the casing 10, multiple drive protrusions 25 proximate to the two limitation extensions 27 and having multiple urge segments 251 formed on multiple predetermined positions of the multiple drive protrusions 25, a forcing face 21 defined on an end of the second casing 20 opposite to the two limitation extensions 27, and a through hole 22 defined on a center of the forcing face 21; wherein the second casing 20 further includes multiple shrinkage trenches 26, and a respective one shrinkage trench 26 is defined between any two adjacent drive protrusions 25, such that when the second casing 20 is moved, the multiple second shrinkage trenches 26 shrink the two tilted contact faces 161 and the two slide tracks 162 of the first casing 10;

3) providing a needle holder 30, wherein the needle holder 30 is defined between the first casing 10 and the second casing 20, and the needle holder 30 including a collection needle 40 accommodated therein, wherein the collection needle 40 corresponds to the through hole 22 of the second casing 20, the needle holder 30 further includes a trigger spring 50 corresponding to the fixing portion 14 of the first casing 10, and a return spring 60 corresponding to the through hole 22 of the second casing 20; wherein the needle holder 30 further includes two symmetrical flexible wings 33 corresponding to the two tilted contact faces 161 of the first casing 10, and the two symmetrical flexible wings 33 include two driving segments 34 formed on two distal ends thereof, wherein the two driving segments 34 force the two symmetrical flexible wings 33 to flexibly retracted inward by using the two tilted contact faces 161 of the first casing 10;

4) abutting the forcing face 21 of the second casing 20 against the collection portion P, and the first casing 10 is pushed toward the collection portion P, wherein the multiple urge segments 251 of the second casing 20 push the two pushed faces 342 of the needle holder 30, the two second inclined guide faces 341 of the needle holder 30 contact with the two tilted contact faces 161 of the first casing 10, and the trigger spring 50 is about to trigger;

5) pushing the two symmetrical flexible wings 33 to flexibly retracted inward by using the two tilted contact faces 161 of the first casing 10 to push the two second inclined guide faces 341 horizontally, wherein the two tilted contact faces 161 and the two slide tracks 162 of the first casing 10 move into the multiple shrinkage trenches 26 of the second casing 20, and the two driving segments 34 abut against the two symmetrical flexible wings 33 of the needle holder 30 to flexibly retract inward so that the two symmetrical flexible wings 33 flexibly retract inward by using the two tilted contact faces 161 of the first casing 10, in the meantime, the trigger spring 50 is released to push the needle holder 30 toward the through hole 22 of the second casing 20, and the syringe needle 41 extends out of the through hole 22 of the second casing 20 and pierces into the collection portion P, wherein the return spring 60 buffers the pushing force of the trigger spring 50 to the syringe needle 41 so as to reduce the stabbing pain.

Thereby, the needle holder 30 includes the trigger spring 50 and the return spring 50 so that when the trigger spring 50 pushes the needle holder 30 toward the through hole 22 of the second casing 20, the return spring 50 buffers the pushing force of the trigger spring 50 to the syringe needle 41 so as to reduce the stabbing pain. Preferably, when the forcing face 21 presses the collection portion P by mating with the protruded rib 222, the subcutaneous fat of the collection portion P is pressed in the thickness so as to reduce the stabbing pain when piercing the collection needle 40 into the skin of the user. In addition, the trigger spring 50 and the return spring 60 pull the needle holder 30 and the syringe needle 41 back quickly to finish the blood collection.

While the preferred embodiments of the invention have been set forth for the purpose of disclosure, modifications of the disclosed embodiments of the invention and other embodiments thereof may occur to those skilled in the art. Accordingly, the appended claims are intended to cover all embodiments which do not depart from the spirit and scope of the invention.

1. A disposable lancing device comprising:
a first casing including a close segment formed on a first end of the first casing, an open segment formed on a second end of the first casing, a slideway formed in the first casing, two tilted contact faces formed on two side wall of the slideway, two first shoulders formed on the slideway, a fixing portion formed on the slideway and corresponding to the close segment, two recesses defined on two side walls of the first casing, and two push fringes formed around the two recesses;
a second casing including an accommodation chamber defined in the second casing, and the second casing being slidably received on the slideway of the first casing, the second casing including two limitation extensions corresponding to the first casing and engaged with the two first shoulders of the casing, multiple drive protrusions proximate to the two limitation extensions and having multiple urge segments formed on multiple predetermined positions of the multiple drive protrusions, wherein a forcing face is defined on an end of the second casing opposite to the two limitation extensions, and a through hole is defined on a center of the forcing face, wherein the through hole has a protruded rib extending outward therearound opposite to the forcing face;
a needle holder defined between the first casing and the second casing, and the needle holder including a collection needle accommodated in the needle holder, wherein the collection needle includes a syringe needle fixed on an end thereof and corresponding to the through hole of the second casing, the needle holder further includes a trigger spring corresponding to the fixing portion of the first casing, and the needle holder further includes a return spring corresponding to the through hole of the second casing; wherein the needle holder includes two symmetrical flexible wings corresponding to the two tilted contact faces of the first casing, and the two symmetrical flexible wings include two driving segments formed on two distal ends thereof, wherein the two driving segments abut against the two symmetrical flexible wings of the needle holder to flexibly retract inward by using the two tilted contact faces of the first casing.

2. The disposable lancing device as claimed in claim 1, wherein two guide faces are formed on the two first shoulders of the first casing, and two first stop faces are formed on the two first shoulders opposite to the two guide faces; wherein the two limitation extensions of the second casing have two first inclined guide faces formed thereon and corresponding to the two guide faces of the two first shoulders, and the two limitation extensions have two second stop faces formed thereon opposite to the two first inclined guide faces, such that the two first inclined guide faces of the two limitation extensions abut against the two guide faces of the two first shoulders, and the two second stop faces of the two limitation extensions move to the two first stop faces of the two first shoulders so that the first casing limits with and slides relative to the second casing.

3. The disposable lancing device as claimed in claim 1, wherein the needle holder further includes a connection projection formed on an end thereof and corresponding to the trigger spring, a first fitting portion extending from an end of the connection projection, a triggering segment formed on an end of the trigger spring and corresponding to the first fitting portion, such that the triggering segment is fixed on the first fitting segment, and the trigger spring includes a positioning segment formed on an end thereof and corresponding to the fixing portion of the first casing, such that the positioning segment is mounted on the fixing portion of the first casing.

4. The disposable lancing device as claimed in claim 1, wherein the needle holder further includes a second fitting portion formed on an end thereof corresponding to the return spring, and the return spring includes a returning segment extending from a first end thereof and corresponding to the second fitting portion, such that the returning segment is fixed on the second fitting portion, and the return spring includes a locating segment extending from a second end thereof and corresponding to the accommodation chamber of the second casing, the accommodation chamber of the second casing has a disposing face corresponding to the locating segment so that the locating segment is fixed on the disposing of the second casing.

5. The air inflatable massage cushion as claimed in claim 2, wherein the first casing further includes two abutting ribs extending from two low ends of the two tilted contact faces, and two slide tracks formed on two high ends of the two tilted contact faces; the two driving segments have two second inclined guide faces formed on two first ends thereof, and the two driving segments have two pushed faces formed on two second ends thereof opposite to the two second inclined guide faces, wherein the second casing includes the multiple shrinkage trenches, and the respective one second shrinkage trench is defined between any two adjacent drive protrusions, such that when the second casing is moved, the multiple second shrinkage trenches shrink the two tilted contact faces and the two slide tracks of the first casing so that when the multiple urge segments of the second casing push the two pushed faces of the needle holder, the two second inclined guide faces are pushed horizontally by the two tilted contact faces of the first casing, the two symmetrical flexible wings of the needle holder are pushed to flexibly retract inward, and the two tilted contact faces and the two slide tracks of the first casing move into the multiple shrinkage trenches of the second casing.

6. The disposable lancing device as claimed in claim 1, wherein a safety sleeve is configured to receive the syringe needle of the collecting needle of the needle holder, the safety sleeve includes a connecting segment formed on a first end thereof, a slot defined in the connecting segment and configured to receive the syringe needle, and a grip portion formed on a second end of the safety sleeve and extending out of the through hole of the second casing.

7. The disposable lancing device as claimed in claim 6, wherein the safety sleeve further includes a stopping portion defined between the connecting segment and the grip portion, a second shoulder defined between the connecting segment and the grip portion of the safety sleeve, and the second shoulder has two extending tabs extending from two sides of the second shoulder, wherein the two extending tabs have a limiting rib facing the grip portion, and the second casing further includes an elongated groove defined in the through hole of the second casing and corresponding to the limiting rib and the two extending tabs so that the limiting rib and the two extending tabs rotate an angle by using the grip portion, and the safety sleeve is engaged in the through hole of the second casing; alternatively, the grip portion rotates an angle so that the limiting rib and the two extending tabs are aligned with the elongated groove of the through hole, and the safety sleeve moves into and out of the through hole.

8. A method of operating the disposable lancing device as claimed in claim 1 comprising steps of:
1) providing a first casing which includes a close segment formed on a first end of the first casing, an open segment formed on a second end of the first casing, a slideway formed in the first casing, two tilted contact faces formed on two surfaces of the slideway, two first shoulders formed on the slideway between the two tilted contact faces and two slide racks, a fixing portion formed on the slideway and corresponding to the close segment, two abutting ribs extending from two low ends of the two tilted contact faces, and the two slide racks formed on two high ends of the two tilted contact faces;
2) providing a second casing which includes an accommodation chamber defined in the second casing, wherein the second casing is slidably received on the slideway of the first casing, the second casing further includes two limitation extensions corresponding to the first casing and engaged with the two first shoulders of the casing, multiple drive protrusions proximate to the two limitation extensions and having multiple urge segments formed on multiple predetermined positions of the multiple drive protrusions, a forcing face defined on an end of the second casing opposite to the two limitation extensions, and a through hole defined on a center of the forcing face; wherein the second casing further includes multiple shrinkage trenches, and a respective one shrinkage trench is defined between any two adjacent drive protrusions, such that when the second casing is moved, the multiple second shrinkage trenches shrink the two tilted contact faces and the two slide racks of the first casing;
3) providing a needle holder, wherein the needle holder is defined between the first casing and the second casing, and the needle holder including a collection needle accommodated therein, wherein the collection needle corresponds to the through hole of the second casing, the needle holder further includes a trigger spring corresponding to the fixing portion of the first casing, and a return spring corresponding to the through hole of the second casing; wherein the needle holder further includes two symmetrical flexible wings corresponding to the two tilted contact faces of the first casing, and the two symmetrical flexible wings include two driving segments formed on two distal ends thereof, wherein the two driving segments force the two symmetrical flexible wings to flexibly retracted inward by using the two tilted contact faces of the first casing;
4) abutting the forcing face of the second casing against the collection portion, and the first casing is pushed toward the collection portion, wherein the multiple urge segments of the second casing push the two pushed faces of the needle holder, the two second inclined guide faces of the needle holder contact with the two tilted contact faces of the first casing, and the trigger spring is about to trigger;
5) pushing the two symmetrical flexible wings to flexibly retracted inward by using the two tilted contact faces of the first casing to push the two second inclined guide faces horizontally, wherein the two tilted contact faces and the two slide racks of the first casing move into the multiple shrinkage trenches of the second casing, and the two driving segments abut against the two symmetrical flexible wings of the needle holder to flexibly retract inward so that the two symmetrical flexible wings flexibly retract inward by using the two tilted contact faces of the first casing, in the meantime, the trigger spring is released to push the needle holder toward the through hole of the second casing, and the syringe needle extends out of the through hole of the second casing and pierces into the collection portion, wherein the return spring buffers the pushing force of the trigger spring to the syringe needle so as to reduce the stabbing pain.

9. A method of operating the disposable lancing device as claimed in claim 2 comprising steps of:
1) providing a first casing which includes a close segment formed on a first end of the first casing, an open segment formed on a second end of the first casing, a slideway formed in the first casing, two tilted contact faces formed on two surfaces of the slideway, two first shoulders formed on the slideway between the two tilted contact faces and two slide racks, a fixing portion formed on the slideway and corresponding to the close segment, two abutting ribs extending from two low ends of the two tilted contact faces, and the two slide racks formed on two high ends of the two tilted contact faces;
2) providing a second casing which includes an accommodation chamber defined in the second casing, wherein the second casing is slidably received on the slideway of the first casing, the second casing further includes two limitation extensions corresponding to the first casing and engaged with the two first shoulders of the casing, multiple drive protrusions proximate to the two limitation extensions and having multiple urge segments formed on multiple predetermined positions of the multiple drive protrusions, a forcing face defined on an end of the second casing opposite to the two limitation extensions, and a through hole defined on a center of the forcing face; wherein the second casing further includes multiple shrinkage trenches, and a respective one shrinkage trench is defined between any two adjacent drive protrusions, such that when the second casing is moved, the multiple second shrinkage trenches shrink the two tilted contact faces and the two slide racks of the first casing;
3) providing a needle holder, wherein the needle holder is defined between the first casing and the second casing, and the needle holder including a collection needle accommodated therein, wherein the collection needle corresponds to the through hole of the second casing, the needle holder further includes a trigger spring corresponding to the fixing portion of the first casing, and a return spring corresponding to the through hole of the second casing; wherein the needle holder further includes two symmetrical flexible wings corresponding to the two tilted contact faces of the first casing, and the two symmetrical flexible wings include two driving segments formed on two distal ends thereof, wherein the two driving segments force the two symmetrical flexible wings to flexibly retracted inward by using the two tilted contact faces of the first casing;
4) abutting the forcing face of the second casing against the collection portion, and the first casing is pushed toward the collection portion, wherein the multiple urge segments of the second casing push the two pushed faces of the needle holder, the two second inclined guide faces of the needle holder contact with the two tilted contact faces of the first casing, and the trigger spring is about to trigger;
5) pushing the two symmetrical flexible wings to flexibly retracted inward by using the two tilted contact faces of the first casing to push the two second inclined guide faces horizontally, wherein the two tilted contact faces and the two slide racks of the first casing move into the multiple shrinkage trenches of the second casing, and the two driving segments abut against the two symmetrical flexible wings of the needle holder to flexibly retract inward so that the two symmetrical flexible wings flexibly retract inward by using the two tilted contact faces of the first casing, in the meantime, the trigger spring is released to push the needle holder toward the through hole of the second casing, and the syringe needle extends out of the through hole of the second casing and pierces into the collection portion, wherein the return spring buffers the pushing force of the trigger spring to the syringe needle so as to reduce the stabbing pain.

10. A method of operating the disposable lancing device as claimed in claim 3 comprising steps of:
1) providing a first casing which includes a close segment formed on a first end of the first casing, an open segment formed on a second end of the first casing, a slideway formed in the first casing, two tilted contact faces formed on two surfaces of the slideway, two first shoulders formed on the slideway between the two tilted contact faces and two slide racks, a fixing portion formed on the slideway and corresponding to the close segment, two abutting ribs extending from two low ends of the two tilted contact faces, and the two slide racks formed on two high ends of the two tilted contact faces;
2) providing a second casing which includes an accommodation chamber defined in the second casing, wherein the second casing is slidably received on the slideway of the first casing, the second casing further includes two limitation extensions corresponding to the first casing and engaged with the two first shoulders of the casing, multiple drive protrusions proximate to the two limitation extensions and having multiple urge segments formed on multiple predetermined positions of the multiple drive protrusions, a forcing face defined on an end of the second casing opposite to the two limitation extensions, and a through hole defined on a center of the forcing face; wherein the second casing further includes multiple shrinkage trenches, and a respective one shrinkage trench is defined between any two adjacent drive protrusions, such that when the second casing is moved, the multiple second shrinkage trenches shrink the two tilted contact faces and the two slide racks of the first casing;
3) providing a needle holder, wherein the needle holder is defined between the first casing and the second casing, and the needle holder including a collection needle accommodated therein, wherein the collection needle corresponds to the through hole of the second casing, the needle holder further includes a trigger spring corresponding to the fixing portion of the first casing, and a return spring corresponding to the through hole of the second casing; wherein the needle holder further includes two symmetrical flexible wings corresponding to the two tilted contact faces of the first casing, and the two symmetrical flexible wings include two driving segments formed on two distal ends thereof, wherein the two driving segments force the two symmetrical flexible wings to flexibly retracted inward by using the two tilted contact faces of the first casing;
4) abutting the forcing face of the second casing against the collection portion, and the first casing is pushed toward the collection portion, wherein the multiple urge segments of the second casing push the two pushed faces of the needle holder, the two second inclined guide faces of the needle holder contact with the two tilted contact faces of the first casing, and the trigger spring is about to trigger;
5) pushing the two symmetrical flexible wings to flexibly retracted inward by using the two tilted contact faces of the first casing to push the two second inclined guide faces horizontally, wherein the two tilted contact faces and the two slide racks of the first casing move into the multiple shrinkage trenches of the second casing, and the two driving segments abut against the two symmetrical flexible wings of the needle holder to flexibly retract inward so that the two symmetrical flexible wings flexibly retract inward by using the two tilted contact faces of the first casing, in the meantime, the trigger spring is released to push the needle holder toward the through hole of the second casing, and the syringe needle extends out of the through hole of the second casing and pierces into the collection portion, wherein the return spring buffers the pushing force of the trigger spring to the syringe needle so as to reduce the stabbing pain.

11. A method of operating the disposable lancing device as claimed in claim 4 comprising steps of:
1) providing a first casing which includes a close segment formed on a first end of the first casing, an open segment formed on a second end of the first casing, a slideway formed in the first casing, two tilted contact faces formed on two surfaces of the slideway, two first shoulders formed on the slideway between the two tilted contact faces and two slide racks, a fixing portion formed on the slideway and corresponding to the close segment, two abutting ribs extending from two low ends of the two tilted contact faces, and the two slide racks formed on two high ends of the two tilted contact faces;
2) providing a second casing which includes an accommodation chamber defined in the second casing, wherein the second casing is slidably received on the slideway of the first casing, the second casing further includes two limitation extensions corresponding to the first casing and engaged with the two first shoulders of the casing, multiple drive protrusions proximate to the two limitation extensions and having multiple urge segments formed on multiple predetermined positions of the multiple drive protrusions, a forcing face defined on an end of the second casing opposite to the two limitation extensions, and a through hole defined on a center of the forcing face; wherein the second casing further includes multiple shrinkage trenches, and a respective one shrinkage trench is defined between any two adjacent drive protrusions, such that when the second casing is moved, the multiple second shrinkage trenches shrink the two tilted contact faces and the two slide racks of the first casing;
3) providing a needle holder, wherein the needle holder is defined between the first casing and the second casing, and the needle holder including a collection needle accommodated therein, wherein the collection needle corresponds to the through hole of the second casing, the needle holder further includes a trigger spring corresponding to the fixing portion of the first casing, and a return spring corresponding to the through hole of the second casing; wherein the needle holder further includes two symmetrical flexible wings corresponding to the two tilted contact faces of the first casing, and the two symmetrical flexible wings include two driving segments formed on two distal ends thereof, wherein the two driving segments force the two symmetrical flexible wings to flexibly retracted inward by using the two tilted contact faces of the first casing;
4) abutting the forcing face of the second casing against the collection portion, and the first casing is pushed toward the collection portion, wherein the multiple urge segments of the second casing push the two pushed faces of the needle holder, the two second inclined guide faces of the needle holder contact with the two tilted contact faces of the first casing, and the trigger spring is about to trigger;
5) pushing the two symmetrical flexible wings to flexibly retracted inward by using the two tilted contact faces of the first casing to push the two second inclined guide faces horizontally, wherein the two tilted contact faces and the two slide racks of the first casing move into the multiple shrinkage trenches of the second casing, and the two driving segments abut against the two symmetrical flexible wings of the needle holder to flexibly retract inward so that the two symmetrical flexible wings flexibly retract inward by using the two tilted contact faces of the first casing, in the meantime, the trigger spring is released to push the needle holder toward the through hole of the second casing, and the syringe needle extends out of the through hole of the second casing and pierces into the collection portion, wherein the return spring buffers the pushing force of the trigger spring to the syringe needle so as to reduce the stabbing pain.

12. A method of operating the disposable lancing device as claimed in claim 5 comprising steps of:
1) providing a first casing which includes a close segment formed on a first end of the first casing, an open segment formed on a second end of the first casing, a slideway formed in the first casing, two tilted contact faces formed on two surfaces of the slideway, two first shoulders formed on the slideway between the two tilted contact faces and two slide racks, a fixing portion formed on the slideway and corresponding to the close segment, two abutting ribs extending from two low ends of the two tilted contact faces, and the two slide racks formed on two high ends of the two tilted contact faces;
2) providing a second casing which includes an accommodation chamber defined in the second casing, wherein the second casing is slidably received on the slideway of the first casing, the second casing further includes two limitation extensions corresponding to the first casing and engaged with the two first shoulders of the casing, multiple drive protrusions proximate to the two limitation extensions and having multiple urge segments formed on multiple predetermined positions of the multiple drive protrusions, a forcing face defined on an end of the second casing opposite to the two limitation extensions, and a through hole defined on a center of the forcing face; wherein the second casing further includes multiple shrinkage trenches, and a respective one shrinkage trench is defined between any two adjacent drive protrusions, such that when the second casing is moved, the multiple second shrinkage trenches shrink the two tilted contact faces and the two slide racks of the first casing;
3) providing a needle holder, wherein the needle holder is defined between the first casing and the second casing, and the needle holder including a collection needle accommodated therein, wherein the collection needle corresponds to the through hole of the second casing, the needle holder further includes a trigger spring corresponding to the fixing portion of the first casing, and a return spring corresponding to the through hole of the second casing; wherein the needle holder further includes two symmetrical flexible wings corresponding to the two tilted contact faces of the first casing, and the two symmetrical flexible wings include two driving segments formed on two distal ends thereof, wherein the two driving segments force the two symmetrical flexible wings to flexibly retracted inward by using the two tilted contact faces of the first casing;
4) abutting the forcing face of the second casing against the collection portion, and the first casing is pushed toward the collection portion, wherein the multiple urge segments of the second casing push the two pushed faces of the needle holder, the two second inclined guide faces of the needle holder contact with the two tilted contact faces of the first casing, and the trigger spring is about to trigger;
5) pushing the two symmetrical flexible wings to flexibly retracted inward by using the two tilted contact faces of the first casing to push the two second inclined guide faces horizontally, wherein the two tilted contact faces and the two slide racks of the first casing move into the multiple shrinkage trenches of the second casing, and the two driving segments abut against the two symmetrical flexible wings of the needle holder to flexibly retract inward so that the two symmetrical flexible wings flexibly retract inward by using the two tilted contact faces of the first casing, in the meantime, the trigger spring is released to push the needle holder toward the through hole of the second casing, and the syringe needle extends out of the through hole of the second casing and pierces into the collection portion, wherein the return spring buffers the pushing force of the trigger spring to the syringe needle so as to reduce the stabbing pain.

13. A method of operating the disposable lancing device as claimed in claim 6 comprising steps of:
1) providing a first casing which includes a close segment formed on a first end of the first casing, an open segment formed on a second end of the first casing, a slideway formed in the first casing, two tilted contact faces formed on two surfaces of the slideway, two first shoulders formed on the slideway between the two tilted contact faces and two slide racks, a fixing portion formed on the slideway and corresponding to the close segment, two abutting ribs extending from two low ends of the two tilted contact faces, and the two slide racks formed on two high ends of the two tilted contact faces;
2) providing a second casing which includes an accommodation chamber defined in the second casing, wherein the second casing is slidably received on the slideway of the first casing, the second casing further includes two limitation extensions corresponding to the first casing and engaged with the two first shoulders of the casing, multiple drive protrusions proximate to the two limitation extensions and having multiple urge segments formed on multiple predetermined positions of the multiple drive protrusions, a forcing face defined on an end of the second casing opposite to the two limitation extensions, and a through hole defined on a center of the forcing face; wherein the second casing further includes multiple shrinkage trenches, and a respective one shrinkage trench is defined between any two adjacent drive protrusions, such that when the second casing is moved, the multiple second shrinkage trenches shrink the two tilted contact faces and the two slide racks of the first casing;
3) providing a needle holder, wherein the needle holder is defined between the first casing and the second casing, and the needle holder including a collection needle accommodated therein, wherein the collection needle corresponds to the through hole of the second casing, the needle holder further includes a trigger spring corresponding to the fixing portion of the first casing, and a return spring corresponding to the through hole of the second casing; wherein the needle holder further includes two symmetrical flexible wings corresponding to the two tilted contact faces of the first casing, and the two symmetrical flexible wings include two driving segments formed on two distal ends thereof, wherein the two driving segments force the two symmetrical flexible wings to flexibly retracted inward by using the two tilted contact faces of the first casing;
4) abutting the forcing face of the second casing against the collection portion, and the first casing is pushed toward the collection portion, wherein the multiple urge segments of the second casing push the two pushed faces of the needle holder, the two second inclined guide faces of the needle holder contact with the two tilted contact faces of the first casing, and the trigger spring is about to trigger;
5) pushing the two symmetrical flexible wings to flexibly retracted inward by using the two tilted contact faces of the first casing to push the two second inclined guide faces horizontally, wherein the two tilted contact faces and the two slide racks of the first casing move into the multiple shrinkage trenches of the second casing, and the two driving segments abut against the two symmetrical flexible wings of the needle holder to flexibly retract inward so that the two symmetrical flexible wings flexibly retract inward by using the two tilted contact faces of the first casing, in the meantime, the trigger spring is released to push the needle holder toward the through hole of the second casing, and the syringe needle extends out of the through hole of the second casing and pierces into the collection portion, wherein the return spring buffers the pushing force of the trigger spring to the syringe needle so as to reduce the stabbing pain.
